(19) United States
(12) Patent Application Publication
(10) Pub. No.: US 2007/0292838 A1
(43) Pub. Date: Dec. 20, 2007

(54) System and method for stimulation and optical surveying in a non invasive manner of the electrical activity of at least a cell

(75) Inventors: Giancarlo Ferrigno, Milano (IT); Sara Mantero, Milano (IT); Alessandra Pedrocchi, Segrate (MI) (IT); Diego Ghezzi, Carate Brianza MI (IT)

Correspondence Address: NIXON & VANDERHYE, PC, 901 NORTH GLEBE ROAD, 11TH FLOOR, ARLINGTON, VA, 22203, US

(73) Assignee: Politecnico di Milano, Milano MI (IT)

(21) Appl. No.: 11/878,528

(22) Filed: Jul. 25, 2007

Related U.S. Application Data
(63) Continuation of application No. PCT/IB2006/000180, filed on Jan. 20, 2006.

(30) Foreign Application Priority Data
Jan. 27, 2005 (IT) MI2005A000114

(51) Int. Cl. C12Q 1/00; C12M 3/00

(52) U.S. Cl. 435/4; 435/287.1

(57) ABSTRACT

This invention relates to an apparatus and a method for optically stimulating and detecting the electrical activity of at least one cell (2A, . . . , 2D) in a cell culture (2) placed on a substrate (3). The apparatus (1) is capable of exciting a bio-activatable molecule (16) positioned in the vicinity of at least one cell (2A, . . . , 2D) through the introduction of a light stimulus in at least one transmission means (5, 5A, . . . , 5D), the latter being located in relation to said at least one cell (2A, . . . , 2D) in such a way that the light stimulus is propagated within said transmission means (5, 5A, . . . , 5D). The apparatus according to the invention has the feature of detecting a light component which is radiated by a fluorescent marker due to the generation of said electrical activity of said at least one cell (2A, . . . , 2D), said light component being propagated through said at least one transmission means (5, 5A, . . . , 5D).

This is a continuation of PCT/IB2006/000180 filed on filed on Jan. 20, 2006 and based, in turn, on Italian Patent Application No. IT MI2005A000114 filed on Jan. 27, 2005.

### BACKGROUND OF THE INVENTION

This invention relates to an apparatus and method for the non-invasive stimulation and optical detection of electrical activity in a cell, in particular an apparatus and method for non-invasive stimulation and optical detection of changes in the electrical potential of at least one cell in a cell culture, in accordance with the pre-characterising clauses of claims 1 and 20.

Apparatus for stimulating and optically detecting the electrical activity of a cell culture, in particular nerve cells, such as for example neurons, are well-known.

The method of operation of such apparatus is based on three different application principles listed below:

I) stimulating and recording the electrical potentials of cells through intra- and/or extra-cellular electrodes, for example through Micro-Electrode Arrays (hereinafter MEA),

II) electrically stimulating the cells through the use of electrodes and recording the electrical potentials generated through fluorescence techniques, for example through the use of fluorescence microscopes and CCD photodetectors or also through an array of photodiodes,

III) optically stimulating the cells through the use of an inactivated “neurotransmitter”, such as glutamate, with subsequent recording of the changes in electrical potentials through intra- and/or extra-cellular electrodes.

Apparatus using these techniques is however affected by a number of problems, among which the most important is the use of electrodes for analysing these cells.

In fact intracellular electrodes are capable of recording a highly selective electrical potential, their accuracy of measurement being the recording of the potential of an individual cell; however, these intracellular electrodes make it impossible to re-use cells, because a cell which has already been examined cannot be examined again using another electrode. In other words the intracellular electrode is invasive and therefore destructive for the cell itself.

In addition to this, because no more than two intracellular electrodes can be inserted per culture, with these instruments it is only possible to investigate the behaviour of individual cells, not the behaviour of entire neuronal networks.

Where extracellular electrodes and MEA are used, there is the advantage that non-invasive operations can be carried out, and different points within the culture can be examined simultaneously, that is the behaviour of an entire neuronal network can be studied. However, extracellular electrodes and MEA are deficient from the point of view of selectivity. In practice they are not very accurate in selecting the cell or cells of interest.

In particular there is the problem that when applying stimulation through an extracellular electrode of a MEA it is certainly possible to excite a cell (or several cells), but it is not possible to avoid repercussions on other cells which are in the vicinity of the electrode. These repercussions may give rise to changes in the measurement of the electrical potential, making the measurement irremediably incorrect.

A similar problem arises when potential is recorded using extracellular electrodes.

In cases where MEA devices are used there is also the problem associated with the electrical fields generated by the electrodes and the lines of signal conduction. In fact the use of MEA devices causes interference between the various lines conducting the electrical signal (also known as the phenomenon of “cross-conduction”), thus generating noise in the signal generated, which makes it not very useful. In addition to this the use of MEA devices generates electrical fields which may influence the electrical activity of the cells surrounding the cell under investigation, and can also alter the electrical interactions between the cells themselves, giving a false measurement of the electrical activity of the cell.

As far as fluorescence detection from excitable cell cultures is concerned, various items of equipment which are capable of performing the function of recording the electrical potential of a cell or a number of cells subjected to investigation are now commercially available.

Equipment designed for the detection of fluorescence from excitable cell cultures is based on the use of CCD television cameras or photodiode arrays which are normally backed-up by an amplification system, filtering, detection and display of the information acquired. In addition to this, such equipment provides for the use of a fluorescence microscope with which the detector can be associated.

However, although such equipment has undoubted advantages, such as for example a high degree of resolution, it has as its main limitation that of high cost of manufacture, and as a consequence a high market cost. In addition to this, such equipment also has the disadvantage that it is unable to incorporate devices for electrically stimulating the cells, in that the microscope channel is used for fluorescence. In fact the system for optically stimulating neurons, in order to be sufficiently resolved, must provide for a high level of focus, which is generally achieved through the use of confocal microscopes.

With this object the use of a CCD device has been proposed to attempt to couple the fluorescence with the confocal microscope. However such equipment only detects fluorescence from the focal volume selected by the microscope and this does not make it possible to detect the fluorescence activity of the entire neuronal network, only the site which has been stimulated.

For example, in the international application having publication number WO00004366A1, a particular type of spectrophotometer which comprises a bundle of three-forked optical fibres capable of being used to detect the fluorescence developed by a cell culture at two different wavelengths is illustrated. The bundle of optical fibres is forked in three ways in that one group of fibres is used to conduct the light to activate the fluorescence, while the remaining groups are used to detect the fluorescence at two different wavelengths.

This device is not capable of investigating networks of neuronal cells on the basis of wholly optical means, as there is no provision for the possibility of stimulating electrical activity through optical stimuli and an inactivated neurotransmitter, it only permits detection of the optical properties of preparations under investigation in a similar way to that provided by a spectrophotometer, but with the feature of working in parallel.

Finally the problem of positioning the culture beneath the microscope needs to be pointed out. In particular, when it is desired to investigate a cell culture it is necessary to meet stringent conditions concerning alignment of the culture in order to guarantee that it is observed correctly.

### SUMMARY OF THE INVENTION

In view of the state of the art described, the purpose of this invention is to provide an apparatus capable of achieving greater selectivity when both stimulating cells and when recording their electrical potentials.

In accordance with this invention this object is achieved through an apparatus and method for stimulating and optically detecting the electrical activity of at least one cell located on a substrate in accordance with the characterising clauses of claims 1 and 16.

Through this invention it is possible to investigate not only a single cell, but even a single portion thereof, or even a complete network of interconnected neurons.

Furthermore, this invention makes it possible to bring about both stimulation and recording of the cell optically, making it possible to achieve an improvement in investigation capacity in comparison with conventional detection equipment.

Moreover, this invention provides a low cost substrate for cell or biological cultures which through integrated optical technology make it possible to investigate cultures deposited without the help of costly equipment.

Another advantage of this invention is in fact that it does not need a microscope to align the culture prepared in such a way that the sample under observation can be known.

Finally, through this invention it is possible to provide an apparatus which, by making use of optical methods, improves the measured signal and increases the selectivity of the examinations.

### BRIEF DESCRIPTION OF THE DRAWINGS

Features and advantages of this invention will be obvious from the following detailed description of a practical embodiment illustrated by way of a non-restrictive example in the appended drawings in which:

FIG. 1 shows a diagrammatical representation of the apparatus according to this invention,

FIG. 2 shows a more detailed illustration of an arrangement of the apparatus in FIG. 1,

FIG. 3 shows a more detailed illustration of another arrangement of the apparatus in FIG. 1,

FIG. 4 shows a diagrammatical illustration of a possible embodiment of this invention in cross-section,

FIG. 5 shows a further diagrammatical representation of the apparatus according to this invention.

### DESCRIPTION OF THE PREFERRED EMBODIMENT

With reference to the appended figures, 1 indicates as a whole apparatus for optical stimulation and detection of the electrical activity of a cell culture 2 placed on a substrate 3.

Stimulation of cell culture 2, for example a culture of nerve cells such as neurons, takes place through a single light stimulus generated by a light source 4 which is propagated through transmission means 5.

These transmission means 5 are located adjacent to the said cell culture 2.

Apparatus 1 comprises a first opto-electronic support 6 and a second opto-electronic support 7 which are optically connected together through transmission means 8.

First opto-electronic support 6 is in optical communication with light source 4, whilst second opto-electronic support 7 is in optical communication with both substrate 3 through said transmission means 5 and with a recording device 9 through further transmission means 10.

These transmission means 9 are capable of recording the electrical activity of cell culture 2.

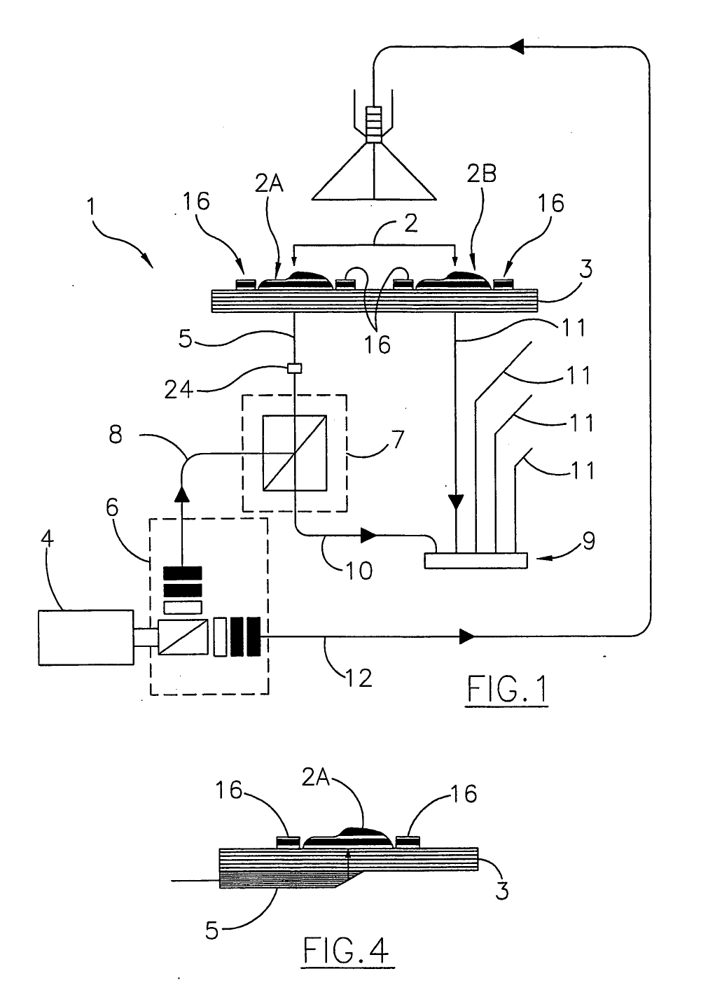

In the embodiment illustrated in FIG. 1, only two cells (or neurons) 2A and 2B are shown for simplicity of illustration.

In this diagrammatical illustration therefore it will be noted that cell 2A is in optical communication with second opto-electronic support 7 through transmission means 5 while cell 2B is in optical communication with recording device 9 through further transmission means 11.

In other words recording device 9 records the electrical activity of cells 2A through the presence of second opto-electronic support 7 and in the case in point can record the electrical activity of cell 2B (or any other cells present in cell culture 2) through a direct optical connection provided by transmission means 11.

Because recording device 9 can record the electrical activity (or electrical membrane potential) of cell 2A or at the same time that of cell 2B, the light stimulus leaving light source 4 must once it has passed through first opto-electronic support 6 be guided through other optical transmission means 12 over cell culture 2 in such a way as to excite a fluorescent marker (not shown in the figures). Advantageously the fluorescent marker is tightly bound to cells 2A and 2B, in particular it is bound to the membrane of cell 2A and 2B. This marker is therefore excited by the stimulus transmitted via transmission means 12 and therefore emits fluorescent light when excited.

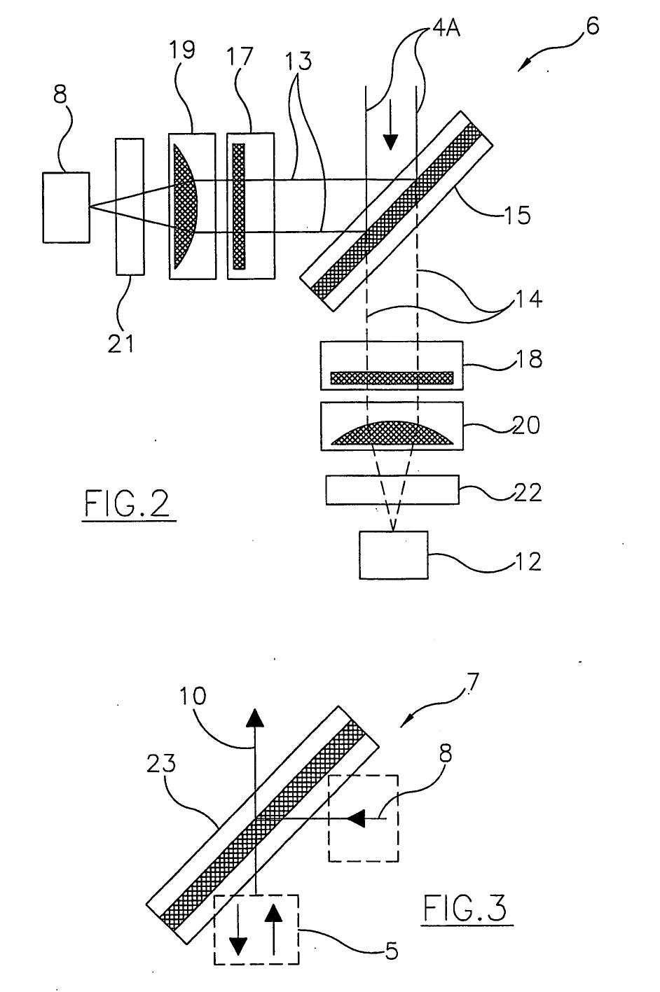

First opto-electronic support 6 is illustrated in its preferred embodiment in FIG. 2. Through this opto-electronic support 6 light stimulus 4A which originates from light source 4 is separated into two spectral components 13 and 14.

This separation takes place through the presence of a dichroic filter 15 which advantageously provides an angle preferably of 45° so as to separate the said light stimulus into the two desired spectral components 13 and 14.

First spectral component 13 is used to activate cell culture 2. This is possible through the presence of light-sensitive bio-activatable molecules 16 in the vicinity of cells 2A and 2B in culture 2. These bio-activatable molecules 16 act as mediators between spectral component 13 and the activation of one or more cells in cell culture 2.

It should be noted that the activation of cell culture 2 takes place in a wholly optical way.

In fact, in the specific embodiment illustrated in FIG. 1, the activation stimulus is sent to cell 2A via transmission means 5 which are in fact positioned in the vicinity of cell 2A itself.

Various types of molecules such as for example inactivated glutamate are commercially available as bio-activatable molecule 16 for exciting cell culture 2. The use of inactivated glutamate, selected for example from the group comprising γ-(CNB-caged), or α-(CNB-caged) or γ-(DMNB-caged), is preferred in this application.

Brief light stimuli in the ultraviolet (UV) field lasting a few tens of milliseconds or less and having a power of less than 20 mW, preferably 10 mW, are necessary in order to activate the glutamate.

Second spectral component 14 is used to excite the fluorescent marker to generate the fluorescence in cell culture 2.

Again in this case it should be noted that excitation of the fluorescent marker in cell culture 2 takes place in a wholly optical way.

In fact in the specific embodiment illustrated in FIG. 1, the activation stimulus will be sent to culture 2 through transmission means 12 which are positioned above the culture.

In the case in point in which cell culture 2 is a culture of biological cells such as neurons first spectral component 13 must for example have a wavelength λ of less than 400 nm, while second component 14 must have, for example, a wavelength λ of between 500 nm and 600 nm.

In order for it to be possible to achieve these wavelength values, each of the two components 13 and 14 must be first filtered, through a stimulation filter 17 for the bio-activatable molecule and an excitation filter 18 for the fluorescence respectively, and then focused, through a pair of lenses 19 and 20 respectively, so that said spectral components 13 and 14 can be guided within corresponding transmission means 8 and 12.

Lens 19, which focuses spectral component 13 of the light pulse within transmission means 8 is for example a lens of the “UV fused silica-plano convex” type, which is a lens suitable for transmission in the ultraviolet (UV) band.

Lens 20, which focuses spectral component 14 of the light pulse within transmission means 12 is for example a lens of the BK7-Plano Convex type.

First opto-electronic support 6 also comprises two optical shutters 21 and 22 which may for example shutters of the electromechanical type which can be controlled through a microcontroller of the PIC type.

These shutters 21 and 22 serve to generate the brief light stimuli required for activation of cell culture 2 and for exciting the fluorescent marker.

Shutters 21 and 22 operate in accordance with predetermined time intervals for opening/closing of the shutters. Each specified time interval may be defined in the relation to the characteristics of the cell culture under examination.

It should be noted that these shutters 21 and 22 also have the function of protecting cell culture 2 from accidental damage due to excessive exposure to light.

Second opto-electronic support 7 is illustrated in its preferred embodiment in FIG. 3.

This opto-electronic support 7 has a dichroic filter 23 which again in this case provides an angle of preferably 45°. This dichroic filter 23 is capable of placing light source 4 in optical communication with cell culture 2 and/or is capable of placing cell culture 2 in optical communication with recording device 9.

The particular configuration illustrated in FIG. 1 therefore makes it possible to both stimulate and record the electrical activity of cell 2A.

In fact thanks to the presence of second opto-electronic support 7 apparatus 1 according to the invention is capable of selecting which cell 2A and/or 2B in cell culture 2 may be stimulated and from which cell 2A and/or 2B electrical activity will be recorded. This is possible because of the presence of a connector 24 provided on transmission means 5, that is located between opto-electronic support 7 and substrate 3.

Advantageously opto-electronic support 7 can be connected to any one of transmission means 11 instead of transmission means 5, such as for example the transmission means relating to cell 2B.

As a result therefore the stage of recording of the electrical potential may take place simultaneously with the stage of stimulation. In fact while a light stimulus, or spectral component 13, is being propagated to stimulate cell culture 2 along transmission means 5, it is possible to record the electrical activity of any cell belonging to cell culture 2 at the same time.

This is possible due to the fact that neurons are cells having electrical activity and this activity is induced by the action of bio-activatable molecule 16, and at the same time the fluorescent marker which is physically coupled to the neuron membrane is excited by spectral component 14.

Thus once the fluorescent marker has been stimulated it emits fluorescent light. This fluorescent emission varies according to the electrical potential of the neuron, at the time when the bio-activatable molecule is activated. The fluorescence emitted via the marker also varies with the change in the electrical activity of the neuron.

It therefore follows that apparatus 1 according to the invention is capable of stimulating the culture and simultaneously detecting a component of the light radiated by the fluorescent marker, the said light component propagating through the same transmission means 5 used to transport the stimulus.

It is specifically as a result of the presence of dichroic filter 23 which is in optical communication with recording device 9 through transmission means 10 that apparatus 1 according to the invention is capable of processing the signal generated by cell 2A and rendering it usable through an amplification and filtering chain (not shown in the appended figures) for subsequent processing.

As already described, this is possible because of second opto-electronic support 7 in that this is located along the optical path of the stimulus for bio-activatable molecule 16, such as for example glutamate. The stimulus activating bio-activatable molecule 16 originating from transmission means 8 is reflected into transmission means 5 corresponding to the cell which has to be stimulated, in this case cell 2A. At the same time as stimulation occurs the fluorescent signal due to generation of the electrical potential of cell 2A is transmitted through dichroic filter 23 of second opto-electronic support 7 and guided to the corresponding photodiode of photodetector 9.

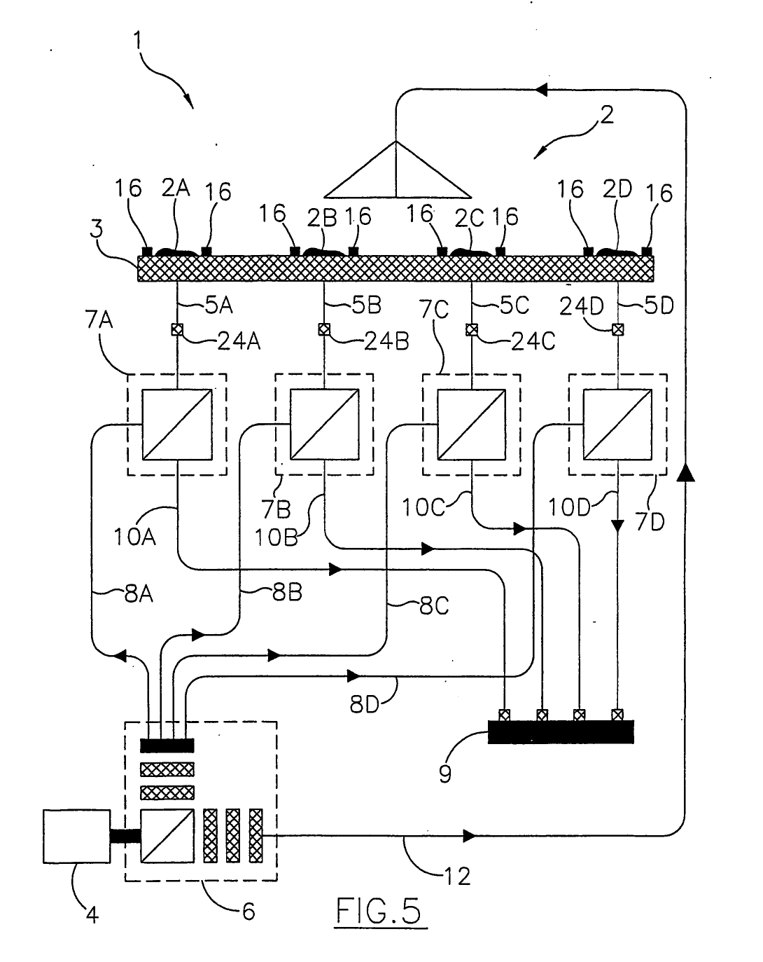

With reference now to FIG. 5, which illustrates another embodiment of this invention in which the components already described are allocated the same numbers, when an array of opto-electronic support 7A, . . . , 7D is provided and there is a corresponding number of cells 2A, . . . , 2D in cell culture 2 which is to be investigated, it should be possible to stimulate one or all of cells 2A, . . . , 2D simultaneously through all transmission means 5A, . . . , 5D by simultaneously making use of the same transmission means for each cell.

In fact it will be seen from FIG. 5 that spectral component 13 is focussed not on a single transmission means, but onto four separate transmission means 8A, . . . , 8D, each of which is in optical communication with a corresponding opto-electronic support 7A, . . . , 7D (it should be noted that the embodiment of these opto-electronic supports 7A, . . . , 7D is wholly equivalent to that shown in FIG. 3).

The presence of connectors 24A, . . . , 24D, each of which is placed between a cell 2A, . . . , 2D and corresponding opto-electronic support 7A, . . . , 7D should also be noted. Through these connectors 24A, . . . , 24D it is possible to decide whether to connect one or all of transmission means 5A, . . . , 5D arriving from substrate 3 to corresponding opto-electronic support 7A, . . . , 7D.

It is therefore possible to define which transmission means 5A, . . . , 5D can be used both to stimulate and detect, or which transmission means 5A, . . . , 5D can be directly connected to recording device 9 by-passing corresponding opto-electronic support 7A, . . . , 7D so as to use that chosen transmission means only for detection and not for stimulation.

In other words an apparatus which is capable of selecting which cell 2A, . . . , 2D of culture 2 to excite and simultaneously record the electrical potential through a single transmission means 5A, . . . , 5D is defined.

Advantageously therefore the transmission means used for stimulation are also used for recording. In other words, each transmission means through which the light stimulus for the bio-activatable molecule is guided also at the same time makes it possible to read the fluorescent signal from the fluorescent marker due to generation of the electrical potential.

Recording device 9 is for example a photodetector, which may comprise an array of a predetermined number of photodiodes or a CCD or CMOS device.

It should be noted that light source 4 is capable of generating both brief light pulses to stimulate the bio-activatable molecule and the light necessary for exciting the fluorescent marker. For example, an arc lamp or a tungsten halogen lamp or a laser source is provided as light source 4.

Transmission means 5, 5A, . . . , 5D, 8, 8A, . . . , 8D, 10, 10A, . . . , 10D, 11 and 12 may for example be optical fibres of the multimodal step index type appropriate for the transmission of UV light, but may also be waveguides incorporated into substrate 3, as illustrated in FIG. 4. This embodiment makes it possible to obtain examination points of smaller size, therefore consistent with dimensions smaller than those of a single neuron.

Use of the aforesaid optical fibres is particularly advantageous in that their transmission interval lies between 200 nm and 900 nm.

In a preferred embodiment the optical fibres have a core of 50 μm, a cladding of 125 μm and an acrylate coating of 250 μm.

Because of the fact that it is possible to create cell cultures 2 according to a predetermined pattern in which the cells are bound to specific areas of substrate 3 it is possible using optical fibres 5 and 11 (embodiment in FIG. 1) or optical fibres 5A, . . . , 5D (embodiment in FIG. 5) to perform rapid investigations not associated with the use of a microscope as it is not necessary to align the culture with the sources and the detectors, as a result of which it is possible to know which cell is stimulated and which cell is responding to the light stimulus at all times.

Substrate 3 is for example a microscope slide, such as to provide a low cost substrate for cell or biological cultures through which the cultures deposited using the apparatus according to the invention can be investigated.

The method for stimulation and optical detection of the electrical activity of at least one cell 2A, . . . , 2D in a cell culture 2 located on substrate 3 according to this invention provides for the stage of generating a light stimulus, that is spectral component 13, capable of exciting bio-activatable molecule 16 positioned in the vicinity of the cell culture. This spectral component 13 is propagated within transmission means 5 which is positioned in relation to at least one cell of said cell culture 2.

There is then a stage of detecting the light component radiated by the fluorescent marker, the said light component being due to generation of the electrical potential, and which is propagated through said transmission means 5.

Advantageously the method according to the invention provides that the stage of detecting the light component radiated by the fluorescent marker can take place simultaneously with the stage of stimulating bio-activatable molecule 16.

Obviously in order to satisfy contingent and specific requirements a person skilled in the art might make many modifications and variants to the configurations described above, all of which will however be within the scope of protection of the invention as defined by the following claims.

1. Apparatus for stimulating and detecting the electrical activity of at least one cell (2A, . . . , 2D) in a cell culture (2), said apparatus (1) comprising:
a substrate (3) on which said at least one cell (2A, . . . , 2D) in said cell culture (2) is placed,
a light source (4) capable of generating a light stimulus (4A) to excite a bio-activatable molecule (16),
at least one first transmission means (5, 5A, . . . , 5D) located in correspondence of said at least one cell (2A, . . . , 2D) in said cell culture (2), in which said light stimulus (4A) is capable of propagating within said at least one first transmission means (5, 5A, . . . , 5D), said bio-activatable molecule (16) being positioned in the vicinity of the said at least one cell (2A, . . . , 2D) in the said cell culture (2),
optical means (6, 7, 7A, . . . , 7D) optically coupled to said at least one first transmission means (5, 5A, . . . , 5D) to stimulate a fluorescent marker, and
recording means (9) capable of recording changes in the fluorescence of the said fluorescent marker, said fluorescent marker being coupled to said at least one cell (2A, . . . , 2D) in said cell culture (2), in which said fluorescence changes are capable of being propagated through said at least one first transmission means (5, 5A, . . . , 5D).

2. Apparatus according to claim 1, wherein said optical means (6, 7, 7A, . . . , 7D) comprise:
a first opto-electronic support (6) capable of separating the said light stimulus (4A) into a first (13) and a second (14) spectral component, said first spectral component (13) being capable of exciting said bio-activatable molecule (16) and said second spectral component (14) being capable of exciting said fluorescent marker, and
at least one second opto-electronic support (7, 7A, . . . , 7D) being capable of stimulating said cell culture (2).

3. Apparatus according to claim 2, characterised in that said first opto-electronic support (6) is in optical communication with said at least one second opto-electronic support (7, 7A, . . . , 7D) through a second transmission means (8, 8A, . . . , 8D).

4. Apparatus according to claim 3, wherein said first opto-electronic support (6) is optically coupled with said light source (4) and with a third transmission means (12), said second spectral component (14) being capable of propagating through said third transmission means (12).

5. Apparatus according to claim 4, wherein said at least one second opto-electronic support (7, 7A, . . . , 7D) is in optical communication with both said substrate (3) and said recording means (9) through said first transmission means (5, 5A, . . . , 5D) and a fourth transmission means (10, 10A, . . . , 10D) respectively.

6. Apparatus according to claim 1, wherein it comprises connector means (24A, . . . , 24D) located between said substrate (3) and said at least one second opto-electronic support (7A, . . . , 7D) capable of connecting at least one of said first transmission means (5A, . . . , 5D) with said recording means (9).

7. Apparatus according to claim 1, wherein said recording means (9) are directly in optical communication with said substrate (3) through fifth transmission means (11).

8. Apparatus according to claim 2, wherein said first opto-electronic support (6) comprises first optical means (15), such as a dichroic filter, to separate said light stimulus (4A) into said first (13) and second (14) spectral component.

9. Apparatus according to claim 2, wherein said first opto-electronic support (6) further comprises filtering means (17, 18) having a stimulation filter (17) to filter said first spectral component (13) and an excitation filter (18) to filter said second spectral component (14).

10. Apparatus according to claim 2, wherein said first opto-electronic support (6) comprises focusing means (19, 20) having lenses to focus said first (13) and second (14) spectral component within said second transmission means (8, 8A, . . . , 8D) and said third transmission means (12).

11. Apparatus according to claim 2, wherein said first opto-electronic support (6) comprises shutters (21, 22) of the electromechanical type capable of generating short pulses of said light stimulus (4A).

12. Apparatus according to claim 2, wherein said at least one second opto-electronic support (7, 7A, . . . , 7D) comprises second optical means (23), such as a dichroic filter.

13. Apparatus according to claim 1, wherein said first transmission means are an optical fibre or a waveguide.

14. Apparatus according to claim 3, wherein said second transmission means are an optical fibre or a waveguide.

15. Apparatus according to claim 4, wherein said third transmission means are an optical fibre or a waveguide.

16. Apparatus according to claim 5, wherein said fourth transmission means are an optical fibre or a waveguide.

17. Apparatus according to claim 6, wherein said fifth transmission means are an optical fibre or a waveguide.

18. Apparatus according to claim 1, wherein said recording means (9) comprise a photodetector having a plurality of photodiodes.

19. Apparatus according to claim 1, wherein said bio-activated molecule (16) is an optically activatable molecule such as a molecule of inactivated glutamate.

20. Method for stimulating and detecting the electrical activity of at least one cell (2A, . . . , 2D) in a cell culture (2), said at least one cell (2A, . . . , 2D) being located on a substrate (3), comprising the stages of:
generating a light stimulus (4A) capable of exciting a bio-activatable molecule (16) and a fluorescent marker, said bio-activatable molecule (16) being positioned in the vicinity of said at least one cell (2A, . . . , 2D) and said fluorescent marker being bound to said at least one cell (2A, . . . , 2D) in said cell culture (2), said light stimulus (4A) being capable of being propagated in at least one transmission means (5, 5A, . . . , 5D), the latter being located in correspondence of said at least one cell (2A, . . . , 2D) in said cell culture (2),
recording changes in fluorescence radiated from said fluorescent marker, said changes in fluorescence being capable of being propagated through said at least one transmission means (5, 5A, . . . , 5D).

21. Method according to claim 20, wherein the stage of recording said changes in fluorescence takes place simultaneously with the stage of generating the light stimulus (4A).

22. Method according to claim 16, wherein the stage of generating a light stimulus (4A) further comprises a stage of separating the light stimulus (4A) into two spectral components (13, 14), said first spectral component (13) being capable of stimulating said bio-activatable molecule (16) and said second spectral component (14) being capable of stimulating said fluorescent marker.

23. Method according to claim 17, wherein the stage of generating a light stimulus (4A) further comprises a stage of separating the light stimulus (4A) into two spectral components (13, 14), said first spectral component (13) being capable of stimulating said bio-activatable molecule (16) and said second spectral component (14) being capable of stimulating said fluorescent marker.

24. Method according to claim 20, wherein it further comprises a stage of selecting whether to connect one or more of said at least one first transmission means (5, 5A, . . . , 5D).
